(19) United States
(12) Patent Application Publication
(10) Pub. No.: US 2010/0324573 A1
(43) Pub. Date: Dec. 23, 2010

(54) SURGICAL CLOSURE INSTRUMENT AND METHODS

(75) Inventors: SOUHAIL TOUBIA, San Juan Capistrano, CA (US); LASZLO GARAMSZEGI, Mission Viejo, CA (US); JUAN MANUEL VELEZ, Ladera Ranch, CA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: SYNOVIS LIFE TECHNOLOGIES, INC., St. Paul, MN (US)

(21) Appl. No.: 12/828,801

(22) Filed: Jul. 1, 2010

Related U.S. Application Data
(63) Continuation of application No. 10/864,135, filed on Jun. 9, 2004, now abandoned.
Application No. 10/864,135 is a continuation-in-part of application No. 10/228,601, filed on Aug. 27, 2002, now abandoned.

(51) Int. Cl. A61B 17/04

(52) U.S. Cl. 606/144

(57) ABSTRACT

Surgical instruments, guides, and methods for closure of fascia and other tissue sites are disclosed. A suture passer guide comprises an elongate body with first and second passages for guiding a suture passer. The long axes of the elongate body, the first passage, and the second passage, preferably lie in three separate parallel planes. A suture passer comprises a housing having a needle tip portion and a first suture grasping surface. An elongate body is located at least partially within the housing and is configured to slide within the housing. The elongate body has a second suture grasping surface at the distal end. The first and second suture grasping surfaces preferably are spaced from the needle tip portion.

### PRIORITY INFORMATION

This application is a continuation-in-part of U.S. patent application Ser. No. 10/228,601 (filed Aug. 27, 2002) and is also based on and claims priority to U.S. Provisional Patent Application No. 60/477,122 (filed Jun. 9, 2003), the entire contents of both of which are hereby expressly incorporated by reference.

### BACKGROUND OF THE INVENTION

1. Field of the Invention

The invention relates to apparatuses, systems and methods for suturing tissue. More particularly, the invention relates to suturing fascia tissue especially during laparoscopic surgery and can include a suture passer and guide.

2. Description of the Related Art

In typical endoscopic or laparoscopic procedures, trocar tubes are inserted into a patient's body through small surgical incisions to create access ports into the body. Thereafter, instruments can be inserted through the access ports to carry out appropriate surgical procedures.

The trocar tubes are removed from the body after the surgical procedures are performed. The incisions are usually sutured closed using a needle, for penetrating the tissue, and a grasper, for handling the suture. The closure procedure can take a significant amount of time. The procedure usually includes identifying the fascia and closing each fascial site with a suture from an external point.

Improperly sutured incisions can lead to bowel herniation through the port sites, as well as the possibility of omental trapping. Incisional hernias can occur in laparoscopic-assisted vaginal hysterectomies and laparoscopic cholecystectomies as well as other advanced laparoscopic procedures.

### SUMMARY OF THE INVENTION

There is a need for apparatuses, systems and methods that reduce operating time and give surgeons direct visualization of fascial and/or peritoneal closings during endoscopic or laparoscopic procedures. Additionally, there is a need for surgical instruments that allow surgeons to control bleeding by rapidly placing sutures around blood vessels of the abdominal wall at the surgical site.

In one embodiment of the invention, a guide device for suturing a tissue comprises an elongate body having a long axis lying within a first plane. A first passage within the body has an inlet opening and an outlet opening along an outer surface of the body. The outlet opening is distal to the inlet opening. A long axis of the first passage lies within a second plane. A second passage within the body has an inlet opening and an outlet opening along the outer surface of the body. The outlet opening is distal to the inlet opening. A long axis of the second passage lies within a third plane. The first, second, and third planes are substantially parallel.

In another embodiment, a medical instrument comprises a housing having a proximal end and a distal end. The housing has a needle tip portion at the distal end and has a first suture grasping surface spaced proximally from the needle tip portion. An elongate body is located at least partially within the housing. The elongate body has a proximal end and a distal end. The elongate body is configured to slide within the housing. The elongate body has a second suture grasping surface at the distal end.

In another embodiment, a medical instrument comprises a housing and an actuator. The actuator is positioned at least partially within the housing. A handle is coupled with a proximal end of the housing and with a proximal end of the actuator. A distal end of the housing comprises a needle tip. A first grasper surface is located on the housing and a second grasper surface is located on the actuator. The first and second grasper surfaces are spaced proximally from the needle tip.

In another embodiment, a medical instrument comprises a housing having a proximal end and a distal end. The housing has a piercing means at the distal end and has a grasping means spaced proximally from the piercing means. An actuating means is coupled with the grasping means and is positioned at least partially within the housing to actuate the grasping means between a closed configuration and an open configuration.

In another embodiment, a medical instrument kit comprises a suture passer guide configured to allow passage of a suture passer through it, and a suture passer. The suture passer comprises a housing having a proximal end and a distal end. The housing has a needle tip portion at the distal end and has a first suture grasping surface spaced proximally from the needle tip portion. An elongate body is located at least partially within the housing having a proximal end and a distal end. The elongate body is configured to slide within the housing. The elongate body has a second suture grasping surface at the distal end.

In another embodiment, a medical instrument kit comprises a suture passer guide and a suture passer configured to pass a suture through the suture passer guide. The suture passer guide comprises an elongate body having a long axis lying within a first plane. A first passage within the body has an inlet opening and an outlet opening along an outer surface of the body. The outlet opening is distal to the inlet opening. A long axis of the first passage lies within a second plane. A second passage within the body has an inlet opening and an outlet opening along the outer surface of the body. The outlet opening is distal to the inlet opening. A long axis of the second passage lies within a third plane. The first, second, and third planes are substantially parallel.

In another embodiment, a medical instrument kit comprises a suture passer guide and a suture passer. The suture passer guide comprises an elongate body having a long axis lying within a first plane. A first passage within the body has an inlet opening and an outlet opening along an outer surface of the body. The outlet opening is distal to the inlet opening. A long axis of the first passage lies within a second plane. A second passage within the body has an inlet opening and an outlet opening along the outer surface of the body. The outlet opening is distal to the inlet opening. A long axis of the second passage lies within a third plane. The first, second, and third planes are substantially parallel. A suture passer comprises a housing having a proximal end and a distal end. The housing has a needle tip portion at the distal end and has a first suture grasping surface spaced proximally from the needle tip portion. An elongate body is located at least partially within the housing. The elongate body has a proximal end and a distal end. The elongate body is configured to slide within the housing. The elongate body has a second suture grasping surface at the distal end.

In another embodiment, a method of passing a suture comprises providing a suture passer having a proximal end and a distal end. The suture passer has a housing and an actuator. The actuator is positioned at least partially within the housing. A handle is coupled with a proximal end of the housing and with a proximal end of the actuator. A distal end of the housing comprises a needle tip. A first grasper surface is located on the housing and a second grasper surface is located on the actuator. The first and second grasper surfaces are spaced proximally from the needle tip. The second grasper surface is positioned near the first grasper surface to hold a portion of a suture outside the patient. The tissue of a patient is pierced at a first location with the needle tip. A portion of the suture is passed into the patient. A portion of the suture is released within the patient. The tissue of a patient is pierced at a second location with the needle tip. A portion of the suture is passed out of the patient.

In another embodiment, a medical instrument comprises a hollow needle having a proximal portion, a distal portion and a notch. The notch is located proximally from a distal tip of the hollow needle. A rod is positioned at least partially within the hollow needle and is movable relative to the notch so as to grasp a portion of a suture within the notch.

In another embodiment, the guide device for suturing a tissue comprises an elongate body having a long axis lying within a first plane; a passage within the body, having an inlet opening and an outlet opening along an outer surface of the body, wherein the outlet opening is distal to the inlet opening, and wherein a long axis of the passage lies within a second plane; wherein the passage is configured to allow passage of a suture therethrough; wherein the first and second planes are substantially parallel; and wherein the long axis of the body and the long axis of the passage are non-parallel.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1a is a side elevational view of an instrument according to one embodiment of the invention.

FIG. 1b is an exploded side elevational view of the instrument of FIG. 1a.

FIG. 2 is a side elevational partial view of a tip of the instrument of FIG. 1a in a closed condition.

FIG. 3 is a side elevational sectional view of the tip of the instrument of FIG. 1a in a closed condition.

FIG. 4 is a side elevational partial view of the tip of the instrument of FIG. 1a in an open condition.

FIG. 5 is a side elevational sectional view of the tip of the instrument of FIG. 1a in an open condition.

FIG. 6 is an isometric view of the tip of the instrument of FIG. 1a in an open condition.

FIG. 7 is another isometric view of the tip of the instrument of FIG. 1a in an open condition.

FIG. 8a is a diagrammatic sketch, partly broken away, of the instrument of FIG. 1a in a closed position, passing a portion of a suture through tissue.

FIG. 8b is a diagrammatic sketch, partly broken away, of the instrument of FIG. 1a in an open position for releasing a portion of the suture.

FIG. 8c is a diagrammatic sketch, partly broken away, of the instrument of FIG. 1a in a closed position passing suture through tissue at another side of the incision and picking up a portion of the suture.

FIG. 8d is a diagrammatic sketch, partly broken away, of the instrument of FIG. 1a pulling a portion of the suture through muscle fascia and peritoneum.

FIG. 8e is a diagrammatic sketch, partly broken away, of the a portion of the suture tied below the skin to complete closure.

FIG. 9a is a side elevational view of another embodiment of an instrument.

FIG. 9b is a perspective view of forceps jaws of the instrument of FIG. 9a in an open position and in a closed position.

FIG. 9c is an isometric view of another embodiment of an instrument in a closed position.

FIG. 9d is a perspective detail view, of a tip of the instrument of FIG. 9c in a closed position.

FIG. 9e is a perspective detail view, of the tip of the instrument of FIG. 9c in an open position.

FIG. 9f is an isometric view of the instrument of FIG. 9c in an open position.

FIG. 10a is a diagrammatic sketch showing one embodiment of a guide coupled with a trocar within the wound to be closed and receiving the instrument of FIG. 1a within a passageway, the instrument carrying a portion of a suture material.

FIG. 10b is a diagrammatic sketch showing the guide of FIG. 10a with the instrument releasing a portion of the suture material.

FIG. 10c is a diagrammatic sketch showing the guide of FIG. 10a with the instrument being received in an opposite and adjacent passageway of the guide for retrieving a portion of the suture material.

FIG. 10d is a diagrammatic sketch showing the guide of FIG. 10a with the instrument pulling a portion of the suture through muscle fascia and peritoneum.

FIG. 10e is a diagrammatic sketch showing the trocar, the guide, and the instrument of FIG. 10a removed from the body and a loop of the suture is ready for wound closure.

FIG. 10f is a diagrammatic sketch, partly broken away, of a portion of the suture tied below the skin to complete closure.

FIG. 11 is a top elevational view of a guide according to one embodiment of the invention.

FIG. 12 is a cross section view of the guide of FIG. 11, taken along line A-A.

FIG. 13 is a top elevational view of the guide of FIG. 11.

FIG. 14 is a front elevational view of the guide of FIG. 11.

FIG. 15 is a cross-sectional view of the guide of FIG. 11, taken along line B-B as shown in FIG. 16.

FIG. 16 is a back elevational view of the guide of FIG. 11.

FIG. 17 is an isometric view of the guide of FIG. 11.

FIG. 18 is an isometric view of a guide according to another embodiment.

FIG. 19 is an isometric view of the guide of FIG. 18 and a trocar.

FIG. 20 is an isometric plan view of another embodiment with a trocar.

FIG. 21 is an exploded isometric view of one embodiment of an instrument.

FIG. 22 is an isometric elevation partial view of a tip of the instrument of FIG. 21.

FIG. 23a is an isometric elevation view of the instrument of FIG. 21 catching a portion of a suture with a needle shaft opening.

FIG. 23b is an isometric elevation view of the instrument of FIG. 21 with a portion of the suture positioned in the needle shaft opening and an inner rod that can be moved for grasping a portion of the suture.

FIG. 23c is an isometric elevation view of the instrument of FIG. 21 with the inner rod postioned near the needle tip for grasping a portion of the suture.

FIG. 24a is an isometric elevation view of a guide according to one embodiment, the guide having a first passageway and a second passageway.

FIG. 24b is an isometric elevation partial cross-sectional view of the guide of FIG. 24a taken along an axis of the first passageway.

FIG. 25a is a diagrammatic sketch of the guide of FIG. 24a receiving the instrument of FIG. 21 within the first passageway, the instrument carrying a portion of a suture.

FIG. 25b is a diagrammatic sketch of the guide of FIG. 24a receiving the instrument of FIG. 21 within the second passageway, the instrument picking up a portion of the suture.

FIG. 26 is a perspective view of a guide according to one embodiment of the invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF THE PREFERRED EMBODIMENT

As should be understood in view of the following detailed description, this application is primarily directed to apparatuses and methods for closing a wound. The apparatuses and methods described below provide for surgical wound closure procedures at surgical locations, preferably during laparoscopic surgery, and provide a variety of tools and instruments useful for wound closure generally. In particular, some embodiments described hereinbelow include closure systems that are particularly well adapted for closing large incisions after removing a trocar. Some embodiments can reduce operating time and give surgeons direct visualization of fascial and/or peritoneal closings during endoscopic or laparoscopic procedures. Additionally, in some embodiments, surgical instruments allow surgeons to control bleeding by rapidly placing sutures around blood vessels of the abdominal wall at a surgical site.

In some embodiments, a suture passer instrument is provided with a piercing tip and a grasping portion spaced from the piercing tip. In some embodiments a guide is provided that can be placed in the wound. The suture passer instrument preferably can pass through the guide and can be directed into the patient at a desired location for performing a surgical wound closure procedure as will be described further below.

Various embodiments of apparatuses and procedures described herein will be discussed in terms of endoscopic or laparoscopic apparatuses and procedures. However, various embodiments may find use in other procedures. As used herein, the term “proximal,” as is traditional, refers to the end portion of an apparatus that is closest to the operator, while the term “distal” refers to the end portion that is farthest from the operator.

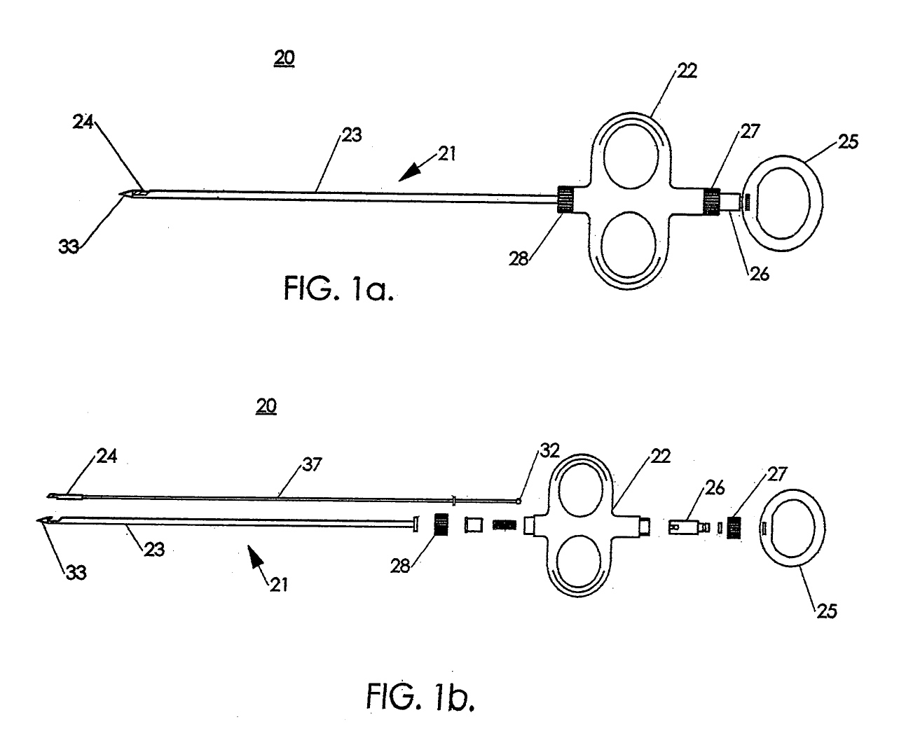

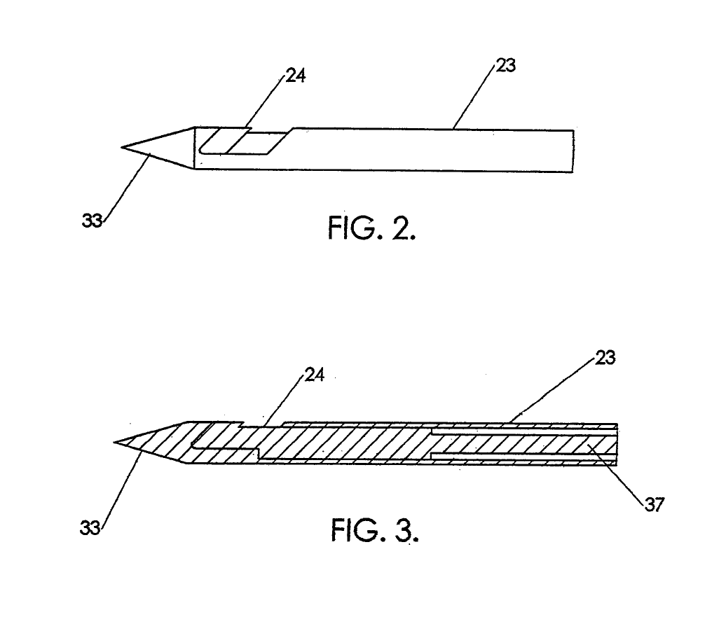

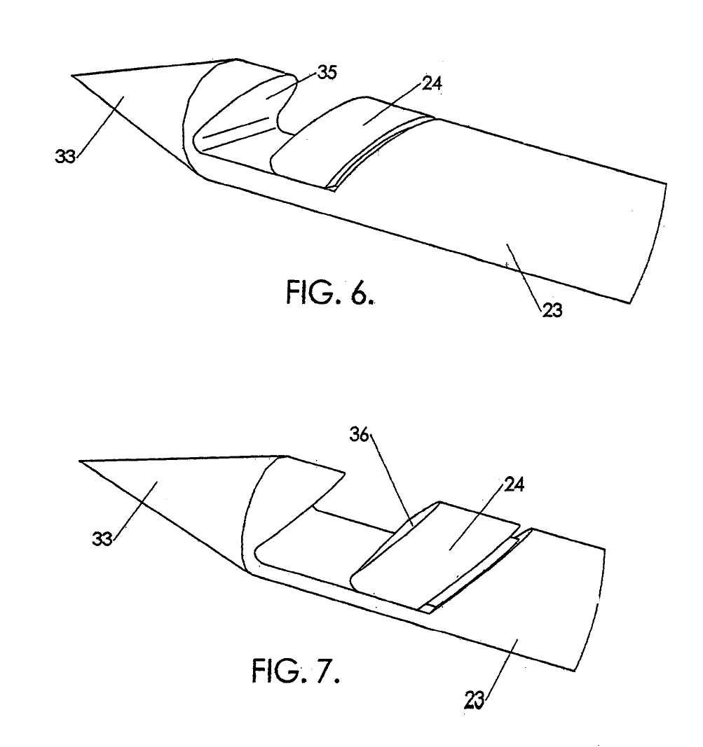

Referring now to FIGS. 1a-7, in one embodiment, a laparoscopic instrument 20 preferably comprises a housing including an elongated outer tube 23. The instrument 20 further comprises an actuating rod 37. The tube 23 and the actuating rod 37 preferably are positioned concentrically about an axis. The rod 37 preferably is positioned at least partially within the outer tube 23. The tube 23 and the rod 37 preferably have grasping surfaces 35 (FIGS. 6) and 36 (FIG. 7) positioned along a distal third of the tube 23 and the rod 37, respectively. The grasping surface 35 preferably is fixed relative to the housing of the instrument 20. The grasping surface 36 preferably is configured to move back and forth within the housing of the instrument 20. The actuating rod 37 preferably is configured to be reciprocated by manipulating an instrument handle 22, and a thumb ring 23. The rod 37 can be actuated in a first direction to drive the grasping surface 36 toward the fixed grasping surface 35 for carrying a portion of a suture. Additionally, the rod 37 can be actuated in a second direction to drive the grasping surface 36 away from the fixed grasping surface 35 for releasing a portion of the suture. As shown in FIGS. 2, 3, 4 and 5, the grasping surfaces 35 and 36 are separate from, and spaced proximally from, a needle tip 33. The tip 33 operates as a sharp needle point that pierces through soft tissue. The sharpness of the needle tip can vary depending on the particular application and preferably is sharp enough to pass through tissue at a desired surgical location. The grasping surfaces 35 and 36 simultaneously grip and pass the suture through the tissue. Providing a needle tip portion on the housing preferably allows the housing to pass easily through the tissue. By providing grasping surfaces that are spaced apart from the needle tip portion, the risk that the grasping surfaces will inadvertently open, or become caught on tissue, during insertion through the tissue will be reduced. Additionally, in some embodiments, locating the grasping surfaces proximally from the needle tip portion can reduce manufacturing costs compared with other devices.

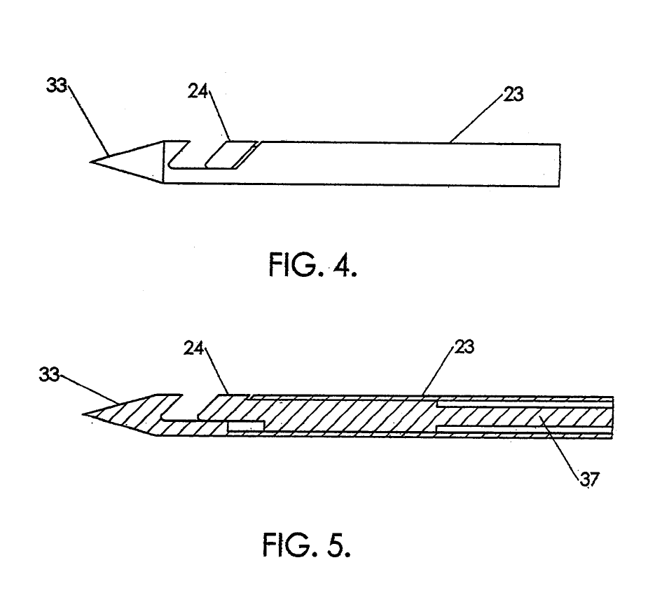

A user, such as, for example, a surgeon, can selectively operate the handle 22 and the thumb ring 23 to move the grasping surface 36 relative to the fixed grasping surface 35 to independently open and close the grasper for carrying or releasing a suture during a laparoscopic operation. In one embodiment, to open the grasper, the surgeon moves the thumb ring 25 connected to the rotating piece 26 forward toward the distal end of the tube 23. FIG. 4 shows the surgical instrument 20 in an open configuration. FIG. 5 shows an inner part 24 of the rod 37 including the grasping surface 36, which is configured to move inside the outer tube 23. FIGS. 2 and 3 show the instrument in a closed configuration.

Various portions or components 21 of the instrument 20 can be detachable if desired, for example, for cleaning or sterilization. As shown in FIG. 1b, in some embodiments, the laparoscopic instrument 20 can be easily disassembled for sterilization. The handle 22 preferably can be separated from the detachable portion 21 by loosening a knurled screw 28 on a fixed handle housing 22, by loosening a knurled screw 27 at a thumb ring 25, and by unlatching a connecting ball 32 from a rotating piece 26. Accordingly, the actuating rod 37 and the tube 23 can be freed from the handle housing 22. By loosening the knurled thumb screw 27, the thumb ring 25 can be disassembled from the fixed handle housing 22 allowing for the cleaning of the inside of the handle-housing area. When disassembled, the parts may be flushed, washed, and dried according to hospital procedures for stainless steel surgical instruments.

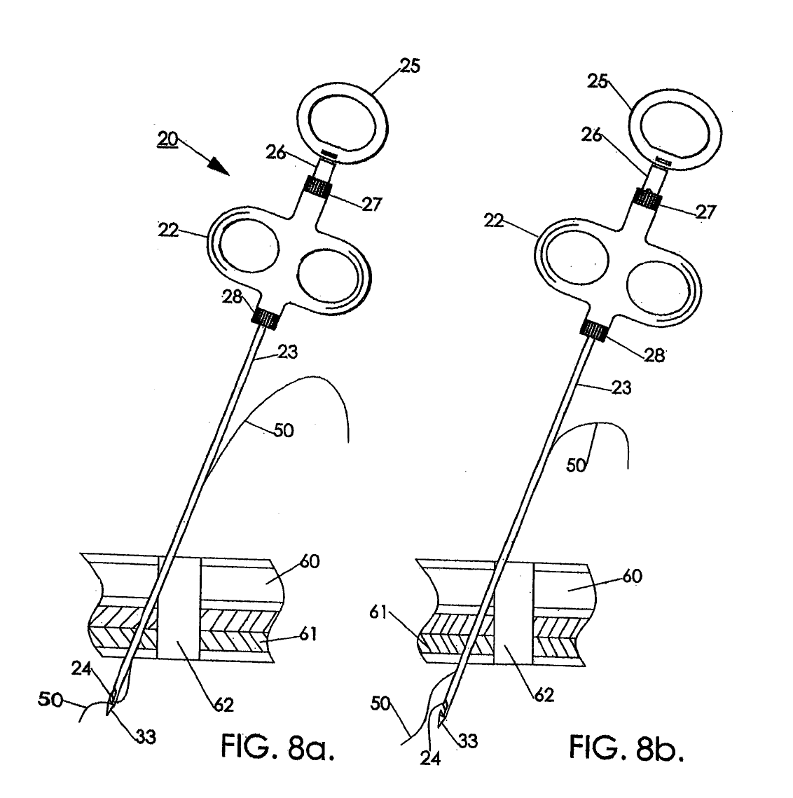

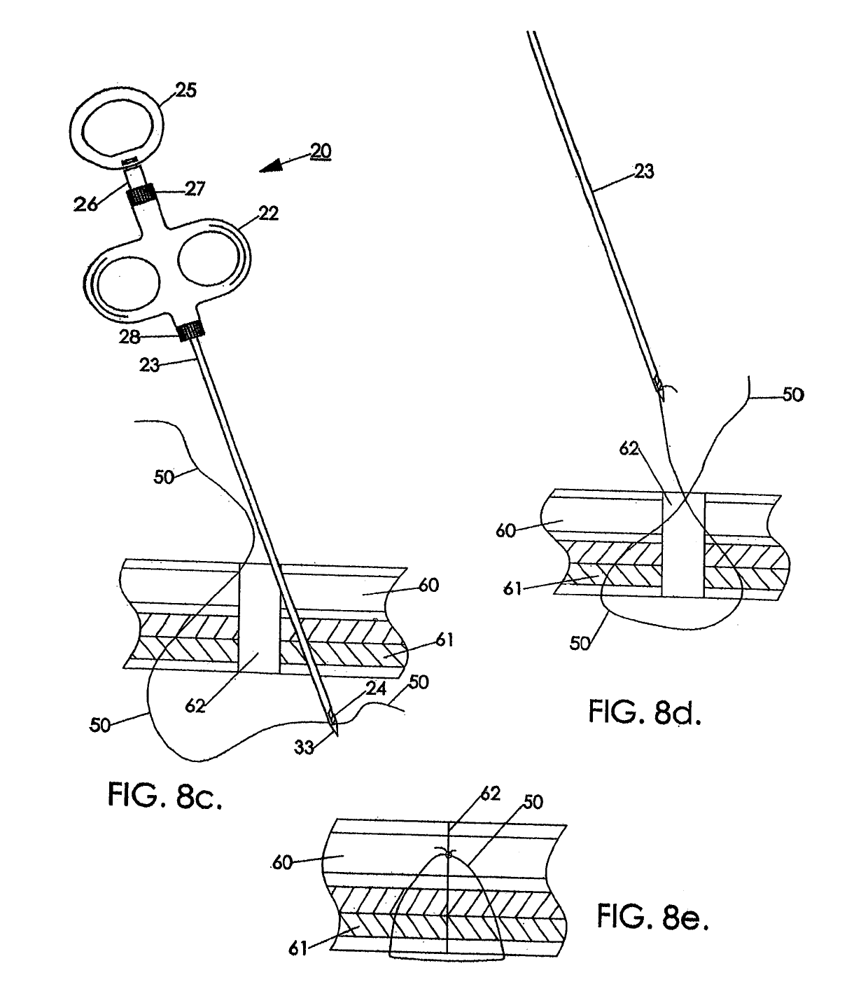

With reference to FIGS. 8a-8e, in one embodiment, a method for passing a suture through soft tissues for closure of an incision 62 during an endoscopic or laparoscopic surgery is provided. As shown in FIG. 8a, a surgeon grasps the suture material 50 with grasping surfaces 35 and 36. The instrument 20, carrying suture material 50, preferably is inserted through the muscle fascia 60 and the peritoneum 61 until the tip 33 and the grasping surfaces 35, 36 are seen through the peritoneum by direct camera visualization, or other suitable means. Subsequently, the surgeon preferably releases the suture 50 within the patient by actuating the moving grasping surface 36, located on the end of the moving inner part 24, away from the fixed grasping surface 35 toward an open configuration. The instrument 20 preferably is withdrawn out of incision 62 as shown in FIG. 8b. With reference to FIG. 8c, the surgeon then inserts the tip 33 of instrument 20 through the muscle fascia 60 and peritoneum 61 at another location, preferably opposite the first point of insertion. Within the patient, the surgeon preferably grasps the suture 50 with the gripping or grasping surfaces 35, 36 and pulls a portion of the suture 50 outside the incision 62 as shown in FIG. 8d. A portion of suture 50 is tied below the skin to complete closure of incision 62 as shown in FIG. 8e.

Materials used to construct the instruments and devices can include surgical stainless steel and other alloys, as well as plastics and other polymers. In one embodiment, a closure system can facilitate camera-viewed laparoscopic procedures. In one embodiment, a closure system can be implemented for laparoscopic port closure. In one embodiment, a closure system can be used in connection with the identification and retraction of ureters during a lympadenectomy. In one embodiment, a closure system can be used in connection with the retraction of kidneys and other structures during laparoscopic nephrectomy. It is contemplated that embodiments of the invention can be used in many other surgical procedures.

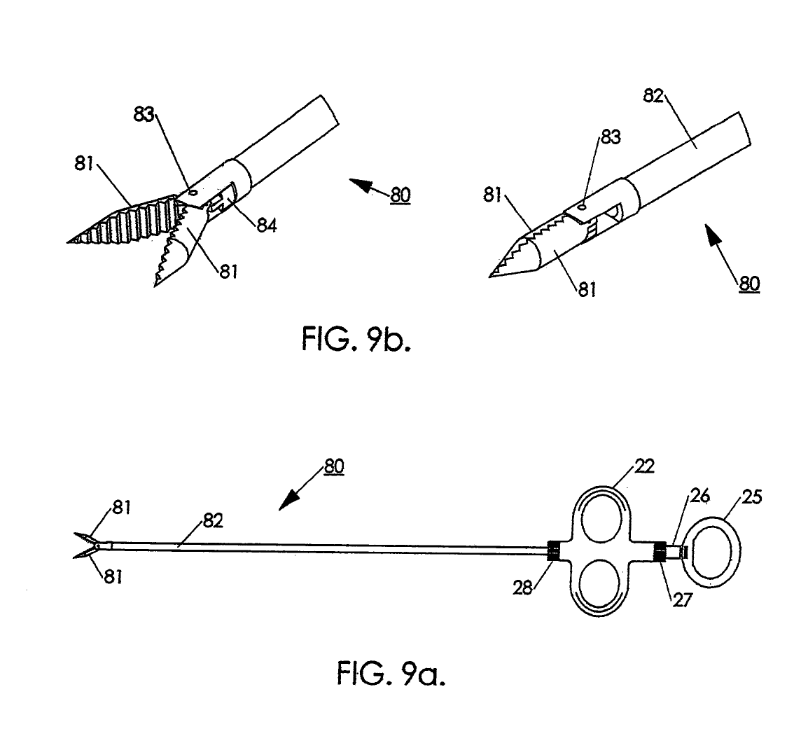

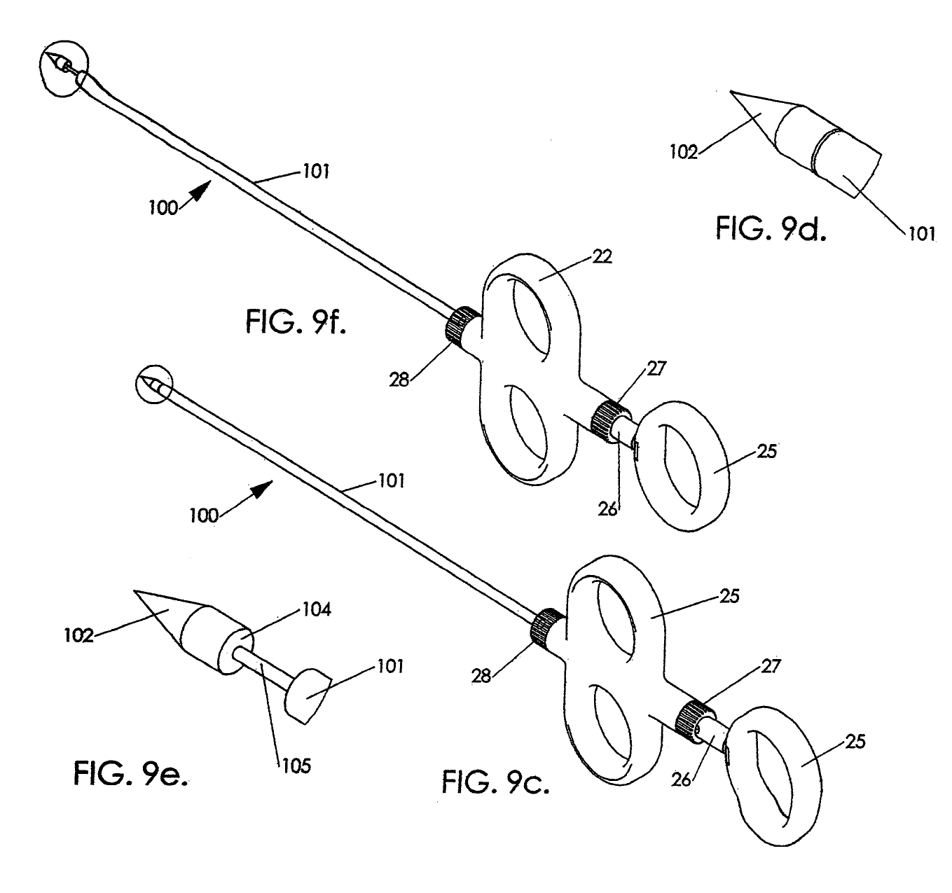

FIGS. 9a through 9f show other embodiments of medical instruments having other advantages. FIG. 9b shows an instrument with an interchangeable grasping forcep shaft 80 with serrated jaws 81 forming a sharp, cone shaped needle tip in a fully closed position. The shaft 80 is connected to a handle 22 and a thumb ring 25 by tightening knurled screws 27 and 28. The interchangeable grasper shaft 80 can have two identical jaws 81 at the tip. The jaws 81 can be retracted by an inner actuating rod 84 to allow the jaws 81 to pierce tissue and carry a suture at the same time. A pin 83 serves as a pivot point for the moving jaws 81. In some embodiments, an exceedingly sharp needle tip formed by the jaws 81 provides for easy penetration of tissue layers. In some cases, the ability to retract both jaws allows the surgeon to more easily manipulate the suture.

As shown in FIG. 9c, another embodiment includes an instrument having a housing shaft portion 100. A shaft 101 preferably is fixed to, and engaged with, a handle 22. An actuating rod 105 preferably includes a needle tip 102 at a distal end. The actuating rod 105 preferably is connected to the handle 22 and a thumb ring 25. The actuating rod 105 preferably moves inside the outer tube or shaft 101. The actuating rod 105 preferably defines a suture gripping area 106 (shown in FIG. 9d) between a grasping surface 104 and a parallel end of the outer tube or shaft 101. One advantage of the illustrated embodiment is that there is no need to rotate the instrument for suture pick-up because the gripping surface is concentrically located on the instrument shaft, allowing suture grasping in 360 degrees. In FIG. 9c, the instrument is shown in a closed position. A detailed view of a closed configuration is shown in FIG. 9d. In FIG. 9f, the instrument is shown in an open position. A detailed view of an open configuration is shown in FIG. 9e.

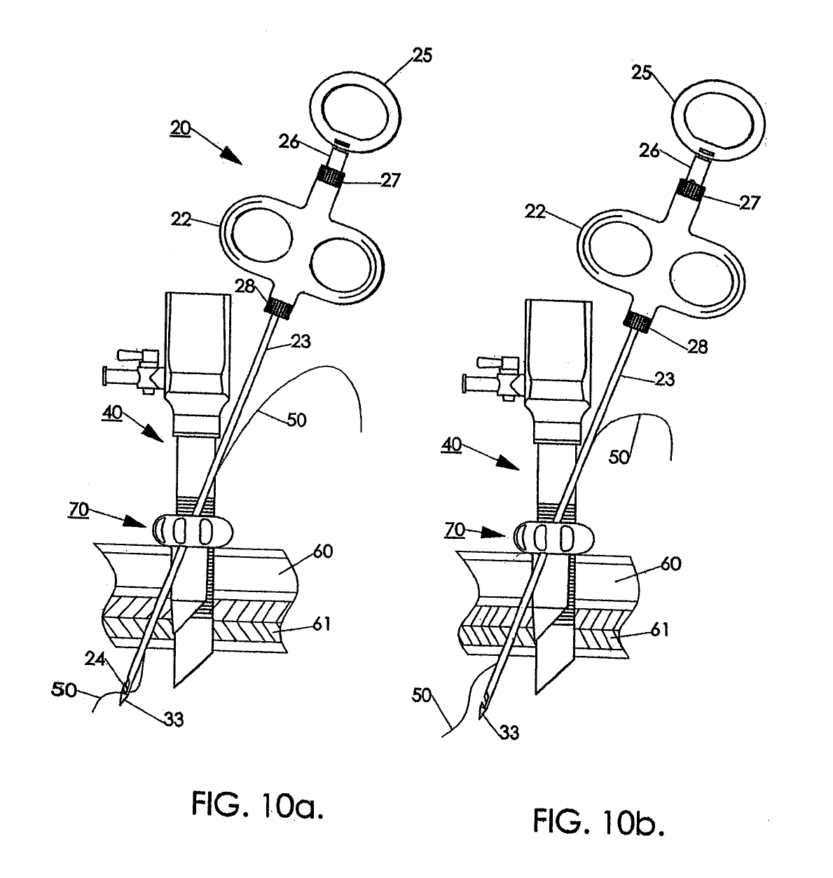

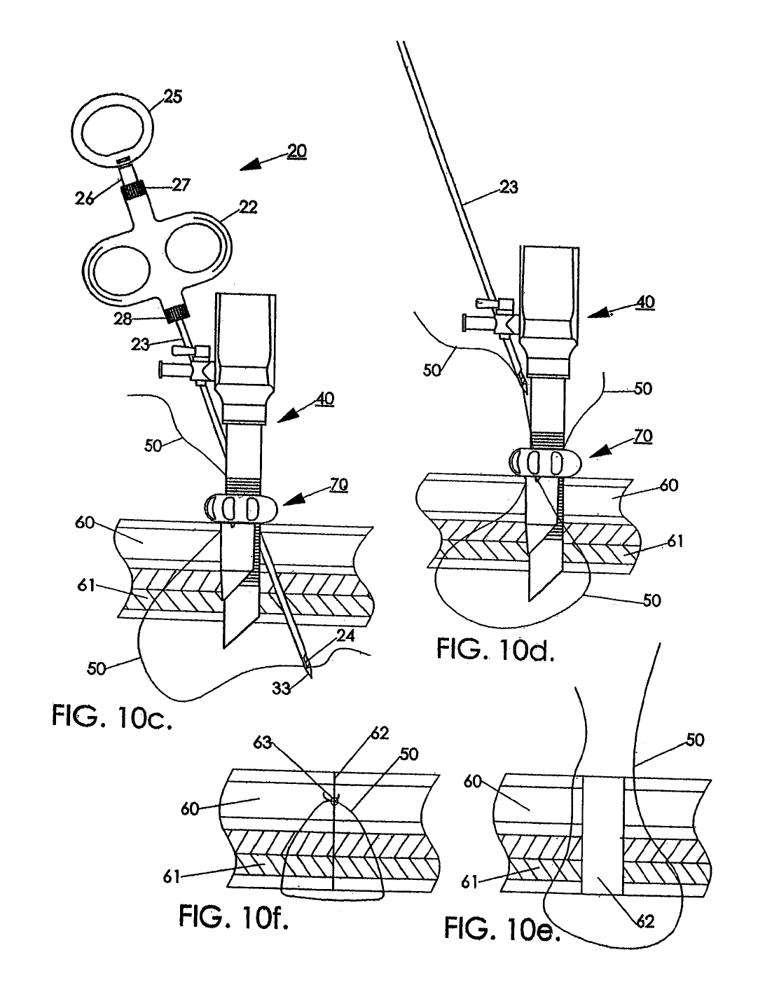

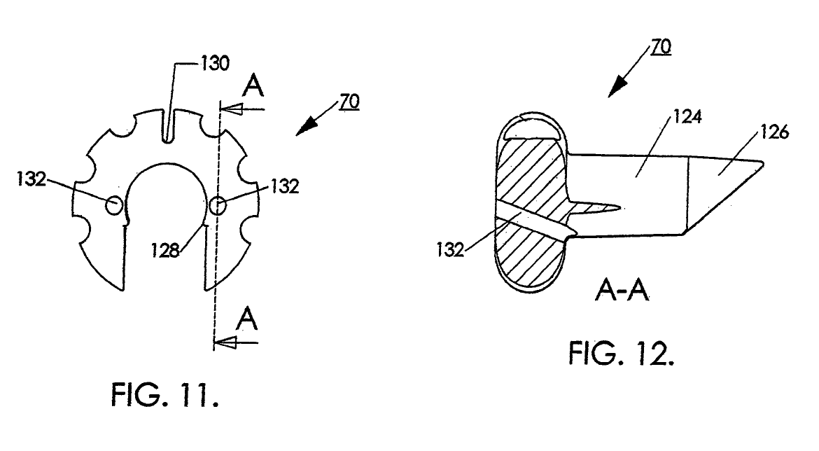

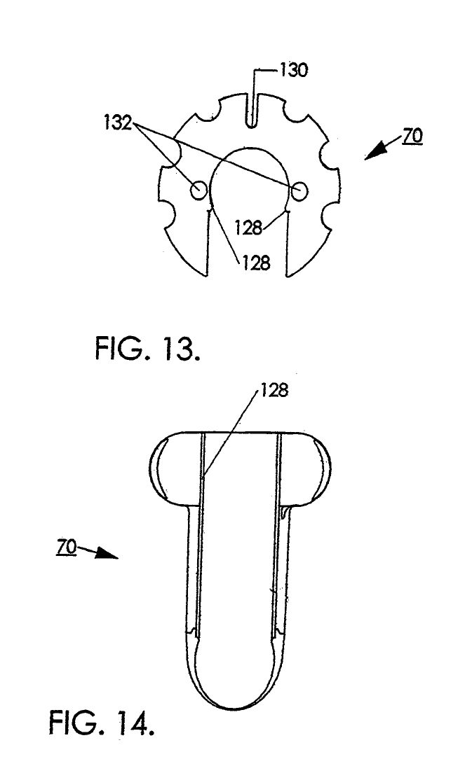

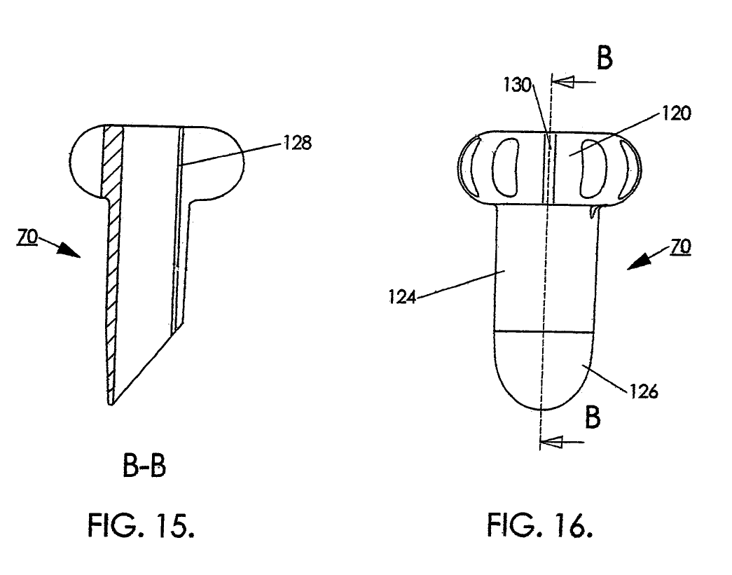

As shown in FIGS. 11-17, in one embodiment, a specially adapted guide 70 can be used in a suturing procedure. One embodiment of an application using a guide 70 is shown in FIGS. 10a-10f. The guide 70 preferably provides for accurately and precisely positioning and removing a suture material 50 into, or from, the patient's body where desired.

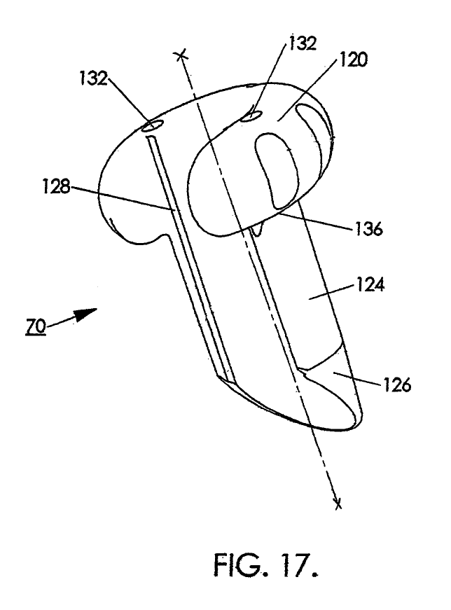

The guide 70 has a longitudinal axis x shown in FIG. 17, and is generally symmetrical about the x-axis. The guide 70 has an extended lip 136 with a radially disposed surface 120, which assists the surgeon in gripping and holding the guide 70. In some embodiments, the gripping surface 120 may be smooth or roughened for easy finger contact.

A surface at the proximal end exposes two passageways 132 through the guide 70. The passageways 132 are parallel to each other, and each forms an angle alpha of approximately 20 degrees with the longitudinal axis x. The two passageways 132 preferably start at the same surface and run in an opposite direction from each other. The diameters of the passageways 132 preferably are sized to receive the surgical instrument to be used.

The guide 70 can have an opening running parallel to the longitudinal axis x. The open side allows the surgeon to attach the guide 70 onto a trocar 40 in some embodiments. In use, the guide 70 can snap onto the shaft of the trocar 40 while it is still positioned in the wound. The extending lip 136 preferably is positioned adjacent to the wound to be sutured. The shaft of the trocar 40 can be concentric with the center opening of the guide 70. The shaft of the trocar 40 preferably can clip into the center opening of the guide 70, between two extruded locking surfaces 128 extending parallel to the longitudinal axis x. A cutout 130 in the guide 70 serves as a stress relief and makes the guide more flexible while attaching onto the trocar 40. The two extruded locking surfaces keep the guide 70 in place in order to complete a skin closure procedure. The lip 136 preferably prevents the guide 70 from sliding further down on the trocar 40 shaft into the wound, and therefore, should be sized to be of a greater diameter than that of the open wound to be sutured. A distal portion 126 of the guide 70 preferably is slightly tapered. Tapering allows for greater ease of insertion while the guide 70 slides into the wound between the trocar 40 and surrounding tissue until it stops at the subcutaneous tissue by the lip portion 136. In some embodiments, the guide 70 can be integrally formed out of high-density polyethylene or other comparable material that is durable and medically inert, and that can serve as a disposable or a reusable product.

As can be seen in FIGS. 10a-10f, the guide 70 can assist in the procedure described above with reference to FIGS. 8a-8e. The guide 70 preferably is attached on the trocar, which is already positioned through the skin incision, muscle, fascia, and peritoneum so that the trocar 40 appears in the view of the laparoscope. The guide 70 can be oriented so that the passageways 132 in the guide 70 are in the required position to complete skin closure.

According to one embodiment, the fascia closure instrument 20, or other suitable instrument as described herein, preferably is inserted with a suture in its grasp through the correctly positioned first passageway 132 in the guide 70 and observed to exit through the peritoneum by laparoscopic view or other suitable manner. The suture can then be released and the instrument 20 withdrawn from the guide 70. The instrument 20 can be placed in the second passageway 132 of the guide and watched by laparoscopic view to exit through the peritoneum. The suture can be grasped by the instrument and withdrawn through the hole made by the instrument 20. The trocar 40 with the guide 70 on its shaft can then be withdrawn from the incision 62 completely. The suture can be tied by standard techniques, thus ensuring that the fascia and peritoneum are closed under the skin.

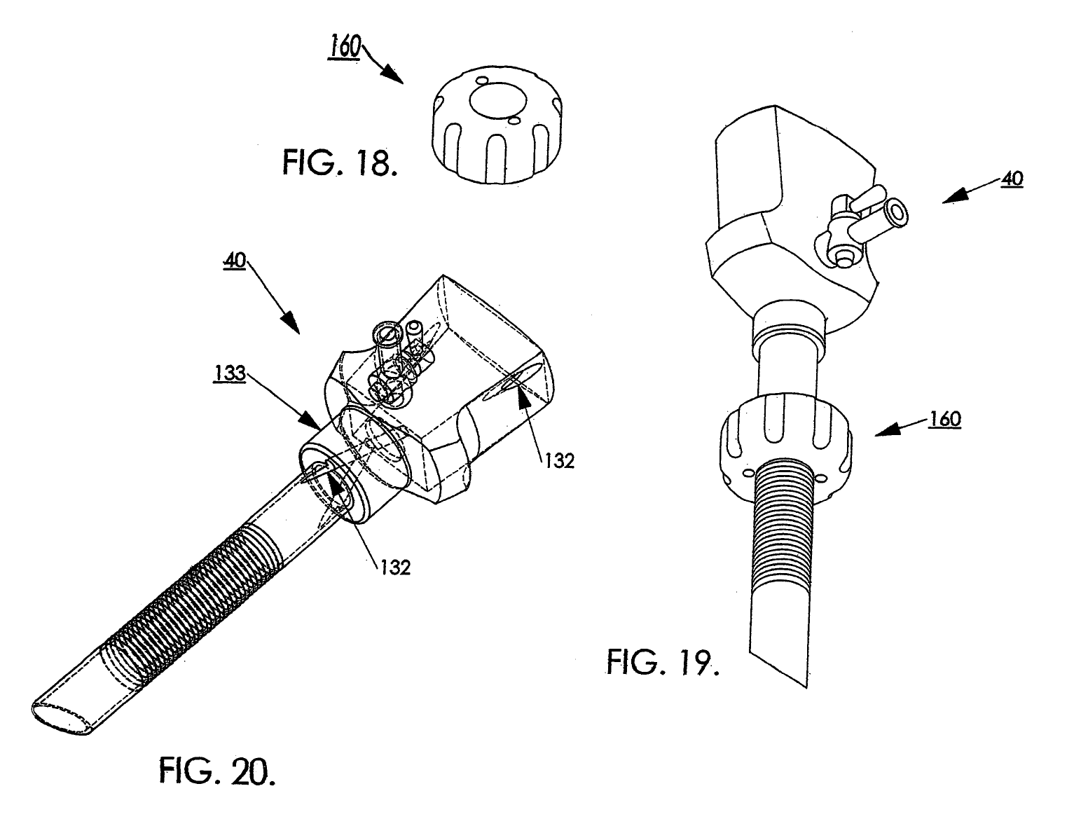

Another embodiment comprises the guide shown in FIGS. 18-19. With reference to FIGS. 11-17, the overall length of the guide shaft 124 may vary, and in some embodiments, the length does not necessarily depend upon the patient's anatomy. In one embodiment, the guide shaft 124 can be eliminated completely. Accordingly, in one embodiment, the guide 160, as shown in FIGS. 18-19, is similar to the extended lip portion 136 of the guide of FIGS. 11-17. As shown in FIG. 19, the guide 160 can be placed on the shaft of the operating trocar 40 before the first surgical incision is performed, assuring that when the laparoscopic surgery is complete, the suture guide will be available immediately to begin tissue closure. Additionally, the suture guide 160 can be sterilized and packaged together with the operating trocars by healthcare manufacturers, offering great economical and surgical advantages.

Another embodiment is shown in FIG. 20, where a suture passer guide ring 133 containing a plurality of passageways is built into the body of an operating trocar. According to the embodiment shown in FIG. 20, a trocar body contains the two passageways 132 and can serve as a suture passer guide.

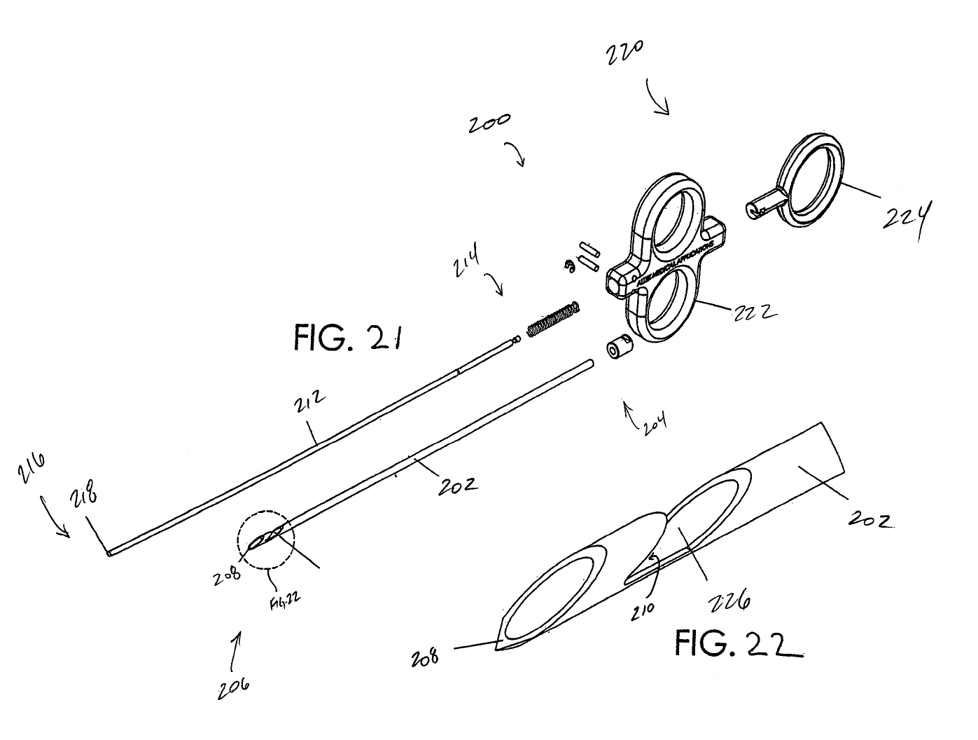

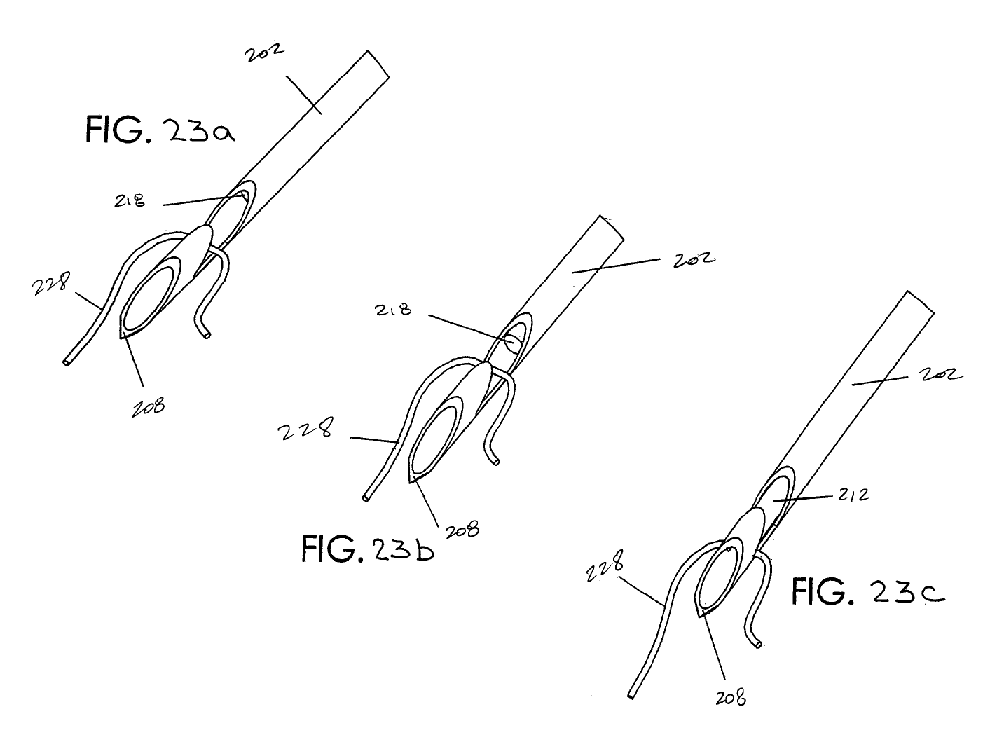

With reference to FIGS. 21-25b, in another embodiment, a medical instrument 200 comprises a housing 202, e.g., an outer tube or shaft, having a proximal end 204 and a distal end 206. The term “housing” is used in its ordinary sense to mean a protective cover designed to contain or support a mechanical component and is a broad term and it includes tubes, shafts, hollow members, and assemblies or parts including a space defined therein for covering another element. The housing 202 preferably has a needle tip portion 208, e.g., a pointed portion, at the distal end 206 and has a first suture grasping surface 210 spaced proximally from the needle tip portion 208. The term “needle” is used in its ordinary sense to mean a pointed implement and is a broad term and it includes devices having varying degrees of sharpness. The term “suture grasping surface” is used in its ordinary sense to mean a surface configured to grasp a suture and is a broad term and it includes surfaces and edges configured to cooperate with other members, surfaces, or edges to hold a suture. An elongate body 212, e.g., an actuator or actuating rod, can be located at least partially within the housing 202. The term “elongate body” is used in its ordinary sense to mean a body having a length greater than a width and is a broad term and it includes guides, medical instruments, and portions thereof, having a length longer than a width. The term “actuator” is used in its ordinary sense to mean a device for moving or controlling something and is a broad term and it includes movable rods coupled within housings to control a grasping surface. The elongate body 212 has a proximal end 214 and a distal end 216. The elongate body 212 preferably is configured to slide within the housing 202. The elongate body 212 preferably is concentrically positioned within the housing 202. The elongate body 212 can have a second suture grasping surface 218 at the distal end 216.

In the illustrated embodiment, the housing 202 is coupled with a handle portion 220, and the elongate body 212 is also coupled with the handle portion 220. In the illustrated embodiment, the handle portion 220 includes a first fixed portion 222 coupled with the housing 202 and a second movable portion 224 coupled with the elongate body 212 to actuate the grasping mechanism of the instrument 200. In some embodiments, the housing 202, the elongate body 212, and the handle portion 220 can be configured to be decoupled as shown in FIG. 21. In other embodiments, a handle portion 222 can be integrally formed with the housing 202 such that the housing 202 comprises the handle portion 222. Additionally, in some embodiments, a handle portion 224 can be integrally formed with the elongate body 212 such that the elongate body 212 comprises the handle portion 224. The handle portion 220 can be any suitable size or shape.

In the illustrated embodiment, the housing 202 of the medical instrument 200 preferably is a hollow needle. The housing 202 has a piercing means at the distal end and has a grasping means spaced proximally from the piercing means. The term “piercing means” is used in its ordinary sense to mean a structure that can be used to force or make a way into or through something and is a broad term and it includes pointed devices, needles, lasers, ultrasonic devices, pneumatic devices, and other structures useful for separating tissue. An actuating means is coupled with the grasping means and is positioned at least partially within the housing 202 to actuate the grasping means between a closed configuration and an open configuration. The term “actuating means” is used in its ordinary sense to mean a structure or device for moving or controlling something and is a broad term and it includes movable rods coupled within housings to control a grasping surface, handle portions, and intermediate elements in an actuating system. The term “grasping means” is used in its ordinary sense to mean structure for taking hold of or seizing firmly and is a broad term and it includes structures, surfaces, and edges for contacting a suture so as to hold the suture for a desired period.

As shown in FIGS. 21-23c, a notch 226 is formed in a side portion of the housing 202. In the illustrated embodiment, the notch 226 is formed in a distal portion of the housing 202. The notch 226 preferably is located proximally from a distal tip 208 of the hollow needle. A portion of the notch 226 defines the first suture grasping surface 210. In some embodiments, the first suture grasping surface 210 is angled relative to an outer surface of the housing 202. As shown in the illustrated embodiment, the first suture grasping surface 210 preferably is fixed relative to the housing 202. The elongate body 212 preferably comprises a rod positioned at least partially within the hollow needle. The rod preferably is movable relative to the notch 226 so as to grasp a portion of a suture 228 within the notch 226.

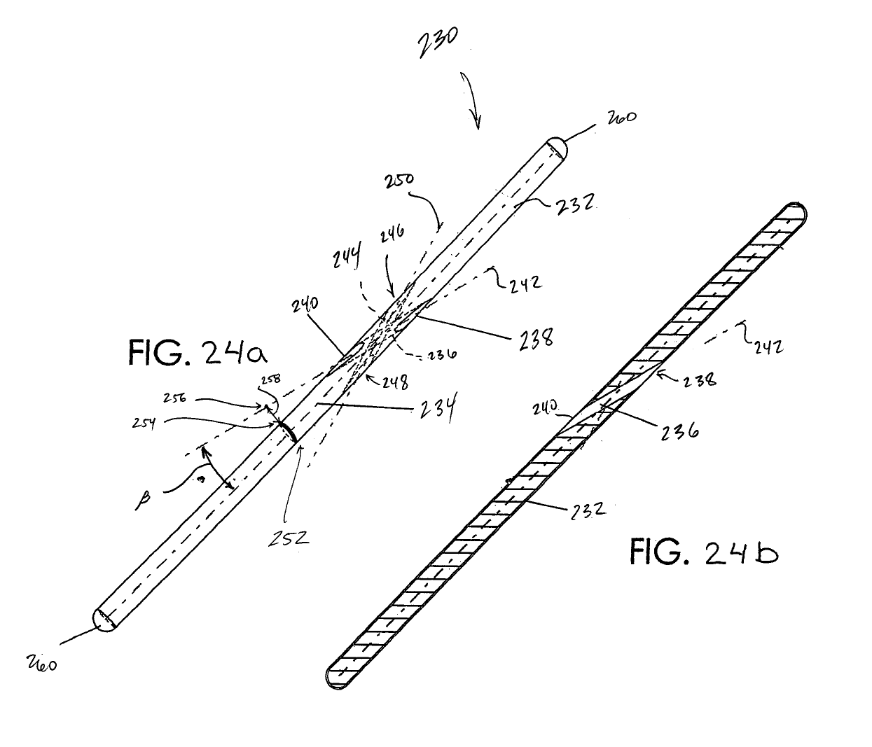

The instrument 200 can have a first configuration wherein the first suture grasping surface 210 is positioned near the second suture grasping surface 218 to grasp a suture 228, as shown in FIG. 23c. The instrument 200 can have a second configuration wherein the first suture grasping surface 210 is spaced from the second suture grasping surface 218 to receive or to release a suture 228, as shown in FIGS. 23a-23b. The instrument 200 has a closed configuration and an open configuration. The elongate body 212 preferably is configured to slide relative to the housing 202. In the closed configuration, the second suture grasping surface 218 is positioned near the first suture grasping surface 210 when the elongate body 212 is in a first position relative to the housing 202. In the open configuration, the second suture grasping surface 218 is spaced from the first suture grasping surface 210 when the elongate body 212 is in a second position relative the housing 202.

In some embodiments, such as, for example, in the embodiment shown in FIGS. 1a-8b, the first suture grasping surface 35 preferably is parallel to the second suture grasping surface 36 in an open configuration. Additionally, in some embodiments, such as that shown in FIGS. 1a-8b, the first suture grasping surface 35 preferably is parallel to the second suture grasping surface 36 in the closed configuration.

With reference to FIGS. 24a-24b, in one embodiment, a guide device 230 for suturing a tissue comprises an elongate body 232 having a long axis 234 lying within a first plane. A first passage 236 within the body 232 has an inlet opening 238 and an outlet opening 240 along an outer surface of the body 232. The outlet opening 240 is distal to the inlet opening 238. A long axis 242 of the first passage 236 lies within a second plane. A second passage 244 within the body 232 has an inlet opening 246 and an outlet opening 248 along the outer surface of the body 232. The outlet opening 248 is distal to the inlet opening 246. A long axis 250 of the second passage 244 lies within a third plane. The first, second, and third planes preferably are substantially parallel. The guide passages 236, 244 can be positioned opposite each other on the guide 230. In the illustrated embodiment, the guide 230 has a first center plane containing the long axis 234 of the body 232, a second plane, on the left of the center plane, containing the axis 242 of the first guide passage 236, and a third plane, on the right of the center plane, containing the axis 250 of the second guide passage 236. Accordingly, the axis of each guide passage lies on one of the offset planes from the center plane of the guide 232. In some embodiments, the guide 230 comprises a plastic rod. Both ends 260 of the guide 230 can be rounded. The guide 230 can have a longer end and a shorter end. In some embodiments, either end 260 can be inserted into the patient for fascia closure, depending on the weight of the patient.

In some embodiments (some not illustrated), the guide device has only one passage. In some of these embodiments the guide device for suturing a tissue comprises an elongate body having a long axis lying within a first plane; a passage within the body, having an inlet opening and an outlet opening along an outer surface of the body, wherein the outlet opening is distal to the inlet opening, and wherein a long axis of the passage lies within a second plane; wherein the passage is configured to allow passage of a suture therethrough; wherein the first and second planes are substantially parallel; and wherein the long axis of the body and the long axis of the passage are non-parallel.

In the illustrated embodiment, the long axes 242, 250 of the first and second passages 236, 244 are non-parallel. An angle beta subtended by the long axis 242 of the first passage 236 and the long axis 234 of the body 232 preferably is about 5 degrees to about 30 degrees. In some embodiments, the angle beta preferably is about 10 degrees to about 20 degrees. In some embodiments, the angle beta is about 15 degrees.

In one embodiment, the body 232 preferably includes a depth indicator 252 on or in the body 232. The indicator 252 preferably is located distal to the outlet openings 240, 248. The depth indicator 252 preferably is positioned circumferentially about the elongate body 232. A plane perpendicular to the long axis 234 of the body 232 passes through a first point 254 on the indicator 252 and passes through a second point 256 along a ray extending along the long axis 242 of the first passage 236. A distance 258 between the outer surface of the body 232 and the second point 256 preferably is less than or equal to about 7 mm. In some embodiments, the distance 258 preferably is less than or equal to about 5 mm.

The guide 230 advantageously positions a medical device 200 for passing a suture into a patient for fascia closure. The depth indicator can be viewed from within the patient using visualization methods to ensure proper placement of the guide 230. Additionally, the guide passages are angled such that the medical device 200 can pass through and grasp a desired amount of tissue from each side of the incision to be held by the suture. Grasping too much tissue can cause bunching in the tissues, potentially leading to significant pain for the patient during recovery. Grasping too little tissue can cause the suture to tear through the tissue in some cases. The depth indicator and predetermined angle positioning of the guide 230 are advantageous because an appropriate amount of tissue can be held by the suture, thereby minimizing the associated pain or trauma that might otherwise be associated with wound closure procedures.

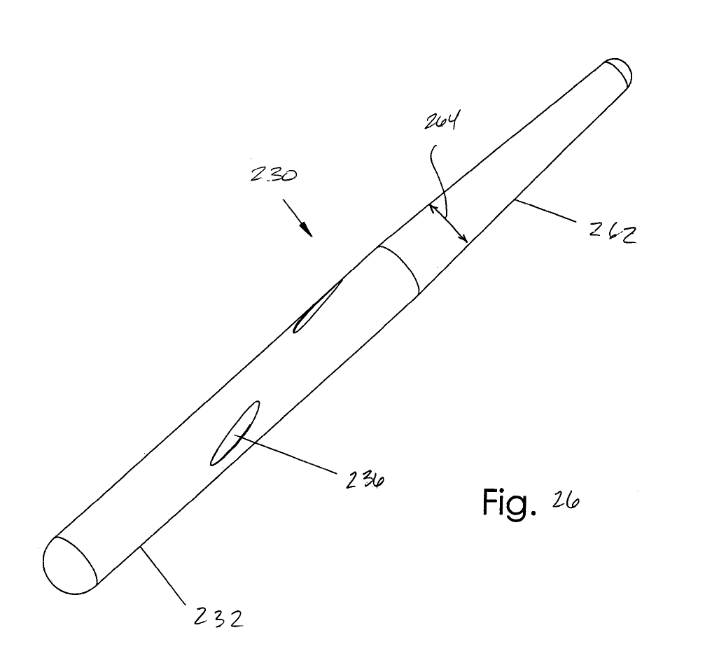

In some embodiments, such as that shown in FIG. 26, the body 232 has a distal tapered section 262. A width 264 of the tapered section 262 preferably tapers from about 20 mm to about 25 mm proximally, to about 10 mm to about 15 mm distally. In some embodiments, a width 264 of the tapered section preferably tapers by about 30% to about 60% from proximal to distal. FIG. 26 illustrates one embodiment of an all-in-one fascia closure guide-rod, incision plug and dilating device. The guide 230 of FIG. 26 preferably includes a plastic rod with one, two, or more guide holes 236, 244. The guide holes 236, 244 are positioned opposite each other on the guide rod 232.

In some cases, surgical instruments for use with bariatric surgery can have a larger size and diameter than other surgical instruments. For example, when surgically treating obesity by performing a gastric bypass operation, the instruments and devices used can require a larger opening or port incision than is required when using average laparoscopic tools. Accordingly, regular operating ports may need to be opened wider to be able to receive oversized surgical instruments. A dilator set is usually used for such a procedure. A dilator typically comprises several instruments with incrementally increasing diameters. The different size dilator instruments have to be placed in, and then removed from, the incision one by one. In some cases gas leakage may occur each time a dilating tool is removed. Additionally, when the wound is dilated, the gas that is used to inflate the abdomen, may leak through the opening relatively quickly. This may require re-inflating the abdominal cavity to be able to continue the surgery.

FIG. 26 illustrates a surgical instrument guide 230 that provides angle guidance for a suture passer device carrying a suture through the fascia, as described above, that can also be used as a plug to stop gas leakage from the wound. Additionally, the guide 230 preferably has one or more tapered ends 262 to be used as a dilating instrument for performing wound dilation. One or both ends of the guide rod can be tapered in a predetermined angle and can be used to widen the surgical wound in order to receive oversized laparoscopic tools.

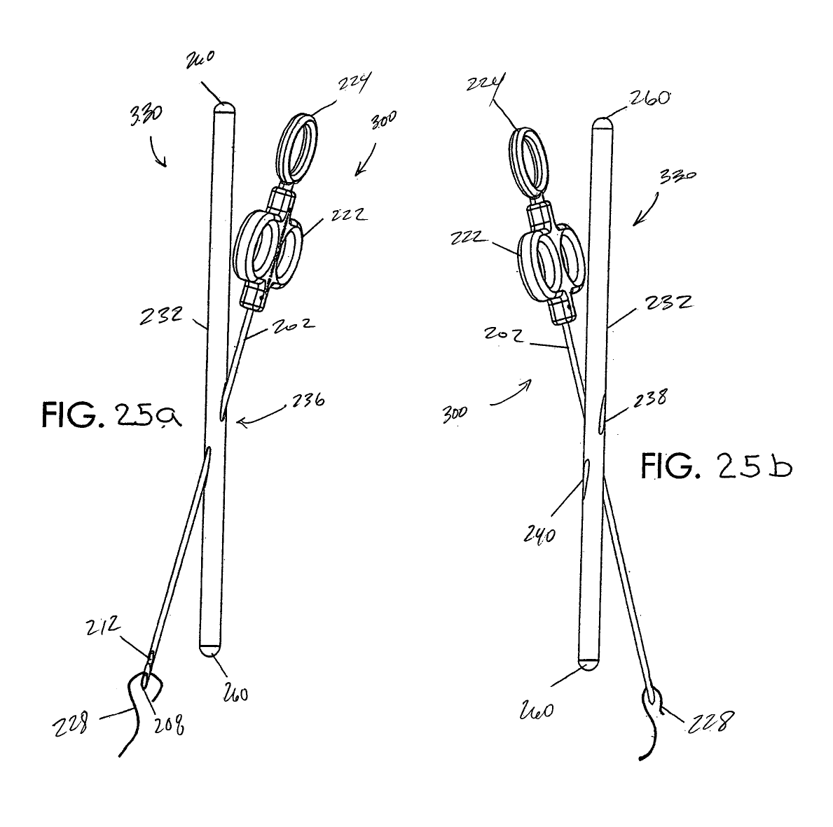

In another embodiment, a medical instrument kit comprises a suture passer guide 330 and a suture passer 300. The suture passer guide 330 preferably is configured to receive and direct the suture passer 300 to a desired position during a surgical wound closure procedure as described herein. The suture passer 300 preferably is configured to pass through the suture passer guide 330. The suture passer 300 and suture passer guide 330 can include instruments and devices as described herein. As shown in FIGS. 25a-25b, the suture passer 300 comprises the medical instrument 200 described in connection with FIGS. 21-23c. As shown in FIGS. 25a-25b, the suture passer guide 330 comprises the guide 230 described in connection with FIGS. 24a-24b.

In another embodiment, a method of passing a portion of a suture 228 comprises providing a suture passer 300, such as, for example, the medical instrument 200 described in connection with FIGS. 21-23c. In some embodiments, a guide 330 can also be provided, such as, for example, the guide 230 described with reference to FIGS. 24a-24b, or the guide 230 described with reference to FIG. 26. The method is similar to the methods described above with respect to FIGS. 8a-8e and FIGS. 10a-10f, except as described below.

According to one embodiment, a trocar 40 preferably is removed from the incision 62 of the patient prior to the insertion of the guide 330. The guide 330 can be directly applied into the opening or surgical incision 62 left by the operating trocar 40. The guide 330 can help the operator place the suture passer 300 into the patient at a predetermined, optimal angle as described herein. The depth of the guide 330 preferably is adjusted so that the tip of the suture passer 300 exits the guide passage below the surface of the skin. The depth indicator preferably can be viewed within the abdomen to determine the appropriate depth of the guide 330. The tissue of a patient preferably is pierced at a first location with a needle tip 208 of the suture passer 300. In some embodiments, the suture passer 300 preferably catches approximately one centimeter of fascia tissue. A portion of the suture 228 can be passed into the patient. The suture passer 300 can be observed in the abdomen using any suitable visualization technique to minimize the risk of injury to surrounding structures. A portion of the suture 228 can be released within the patient. The suture passer 300 preferably is withdrawn and repositioned. The tissue of the patient preferably is pierced at a second location with the needle tip 208. In some embodiments, the instrument shaft 202 with the needle tip 208 is attached to a handpiece 220 allowing the operator to control the grasping surface 218 located at the end of the retractable inner rod 212. In some embodiments, the suture 228 can be positioned into the opening 226 on the needle shaft 202 after piercing tissue layers so that it can be grasped by fully retracting the inner rod 212. A portion of the suture 228 is passed out of the patient. The suture 228 can be configured in an intra-abdominal loop outside the abdomen. The suture passer guide 330 can be removed from the wound. A knot can be tied in a portion of the suture 228 below the skin level to close the fascial defect within the abdominal wall. The surgeon preferably checks the wound to assure proper placement of the suture.

The systems described herein preferably are capable of being used for the closure of fascia and other laparoscopic procedures that use large operative ports or trocars. The systems and methods preferably reduce the risk of postoperative hernia defects and also can be used to stop acute bleeding from the abdominal wall.

The various devices, methods, procedures, and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

1. A guide device for suturing a tissue, the device comprising:
an elongate body having a long axis lying within a first plane;
a first passage within the body, having an inlet opening and an outlet opening along an outer surface of the body, wherein the outlet opening is distal to the inlet opening, and wherein a long axis of the first passage lies within a second plane;
a second passage within the body, having an inlet opening and an outlet opening along the outer surface of the body, wherein the outlet opening is distal to the inlet opening, and wherein a long axis of the second passage lies within a third plane;
wherein the first, second, and third planes are substantially parallel and offset from each other.

2. The device of claim 1, wherein the long axes of the first and second passages are non-parallel.

3. The device of claim 1, wherein the body further comprises a distal tapered section.

4. The device of claim 1, further comprising a depth indicator on or in the body, wherein the indicator is located distal to the outlet openings.

5. The device of claim 1, wherein an angle subtended by the long axis of the first passage and the long axis of the body is about 5° to about 30°.

6. The device of claim 5, wherein an angle subtended by the long axis of the first passage and the long axis of the body is about 10° to about 20°.

7. The device of claim 6, wherein an angle subtended by the long axis of the first passage and the long axis of the body is about 15°.

8. The device of claim 3, wherein a plane perpendicular to the long axis of the body passes through a first point on the indicator and passes through a second point along a ray extending along the long axis of the first passage; and
wherein a distance between the outer surface and the second point is less than or equal to about 7 mm.

9. The device of claim 8, wherein a distance between the outer surface and the second point is less than or equal to about 5 mm.

10. The device of claim 3, wherein a width of the tapered section tapers from about 20 to about 25 mm proximally to about 10 to about 15 mm distally.

11. The device of claim 3, wherein a width of the tapered section tapers by about 30% to about 60% from proximal to distal.

12. The device of claim 4, wherein the depth indicator is positioned circumferentially about the elongate body.

13-43. (canceled)

44. A guide device for suturing a tissue, the device comprising:
an elongate body having a long axis lying within a first plane;
a passage within the body, having an inlet opening and an outlet opening along an outer surface of the body, wherein the outlet opening is distal to the inlet opening, and wherein a long axis of the passage lies within a second plane;
wherein said passage is configured to allow passage of a suture therethrough;
wherein the first and second planes are substantially parallel and offset from each other; and wherein the long axis of the body and the long axis of the passage are non-parallel.

45. A guide device according to claim 1 wherein the first and second passages are substantially linear.

46. A guide device according to claim 44 wherein the long axis of the first plane and the long axis of the first passage are substantially linear.
